(19) United States
(12) Patent Application Publication
(10) Pub. No.: US 2025/0177747 A1
(43) Pub. Date: Jun. 5, 2025

(54) DEVICES, SYSTEMS AND METHODS FOR PERSONALIZED NEUROMODULATION

(71) Applicants: Yingchun ZHANG, Katy, TX (US); Feng FANG, Houston, TX (US)

(72) Inventors: Yingchun ZHANG, Katy, TX (US); Feng FANG, Houston, TX (US)

(21) Appl. No.: 18/845,086

(22) PCT Filed: Mar. 7, 2023
(86) PCT No.: PCT/US2023/014674

§ 371 Date: Sep. 9, 2024

Related U.S. Application Data
(60) Provisional application No. 63/318,887, filed on Mar. 11, 2022.

(51) Int. Cl. A61N 1/36; A61N 1/04

(52) U.S. Cl. CPC A61N 1/36031; A61N 1/0456

(57) ABSTRACT

A system for personalized neuromodulation includes a neuroimaging recording device including sensors for detecting neural activity in a patient. An optimization module determines a personalized neuromodulation treatment protocol for the patient. The optimization module includes a neuromodulation target optimization module for determining neural targets in the patient for the personalized neuromodulation treatment protocol. The optimization module includes a neuromodulation parameters optimization module for determining optimized neural stimulation parameters for the personalized neuromodulation treatment protocol. A personalized neural stimulation device receives the neural targets from the neuromodulation target optimization module and receives the optimized neural stimulation parameters from the neuromodulation parameters optimization module. The personalized neural stimulation device delivers the personalized neuromodulation treatment protocol to the patient. The personalized neural stimulation device includes at least one neural stimulation device for stimulating the targets in the patient. A neuroimaging navigation system determines a placement of the neural stimulation device(s) about the patient.

### CROSS-REFERENCE TO RELATED APPLICATIONS

The present application claims the benefit of U.S. Provisional Patent Application No. 63/318,887, filed on Mar. 11, 2022, the entire contents of which are hereby incorporated herein by reference.

### FIELD

The present disclosure relates to neuromodulation, and more specifically, to devices, systems and methods for personalized neuromodulation.

### BACKGROUND

Neuromodulation refers to altering nerve activity through targeted delivery of a stimulus, such as an electrical stimulus. Neuromodulation can be employed to normalize or modulate nervous tissue function. Neuromodulation is a developing therapy that can employ a range of electrical or electromagnetic stimuli such as electrical current, a magnetic field, or a drug/pharmaceutical compound delivered directly to an area of interest (e.g., in a patient's brain).

Neuromodulation, whether electrical or magnetic, can utilize the body's natural biological response by stimulating nerves or neurons, thus influencing populations of interconnected nerves or neurons by releasing neurotransmitters (e.g., dopamine), or other chemical messengers to modulate the excitability and firing patterns of the corresponding neural circuits.

Neuromodulation is therapeutically effective in treating numerous neurological and psychiatric diseases. However, due to the patient heterogeneity, neuromodulation treatment outcomes are often highly variable, requiring patient-specific stimulation targets and parameters by accommodating inherent variability and intersession alteration during treatments.

### SUMMARY

Provided in accordance with aspects of the present disclosure is a system for personalized neuromodulation including a neuroimaging recording device including sensors for detecting neural activity in a patient. An optimization module determines a personalized neuromodulation treatment protocol for the patient. The optimization module includes a neuromodulation target optimization module for determining at least one neural target in the patient for the personalized neuromodulation treatment protocol. The optimization module includes a neuromodulation parameters optimization module for determining optimized neural stimulation parameters for the personalized neuromodulation treatment protocol. A personalized neural stimulation device receives the neural target(s) from the neuromodulation target optimization module and receives the optimized neural stimulation parameters from the neuromodulation parameters optimization module. The personalized neural stimulation device delivers the personalized neuromodulation treatment protocol to the patient. The personalized neural stimulation device includes at least one neural stimulation device for stimulating the target(s) in the patient. A neuroimaging navigation system determines a placement of the neural stimulation device(s) about the patient.

In an aspect of the present disclosure, the neural stimulation device includes a stimulation device, such as a transcranial stimulation device.

In an aspect of the present disclosure, the neural stimulation device is a transcranial magnetic stimulator (TMS), a transcranial direct current stimulator (tDCS), a transcranial electrical stimulator (tES), a transcranial focused ultrasound (tFUS), a deep brain stimulator (DBS), a intracranial cortical stimulation (ICS), a pressure/mechanical wave (shock wave) stimulator, a micro-scale magnetic fields stimulator, an optogenetics stimulator, or a photobiomodulation stimulator.

In an aspect of the present disclosure, the neuroimaging recording device is an electroencephalogram (EEG) system, a functional magnetic resonance imaging (fMRI) system, a diffusion tensor imaging (DTI) system, a positron emission tomography (PET) system, a magnetic resonance spectroscopy (MRS), a Single-photon emission computed tomography (SPECT) system, a functional ultrasound imaging (fUS) system, a computer tomography (CT) system, a functional near-infrared spectroscopy (fNIRS) system, or a magnetoencephalography (MEG) system.

In an aspect of the present disclosure, the neuroimaging recording device includes wired EEG sensors.

In an aspect of the present disclosure, the neuroimaging recording device includes wireless EEG sensors.

Provided in accordance with aspects of the present disclosure is a method of personalized neuromodulation including detecting neural activity in a patient. The method includes determining a personalized neuromodulation treatment protocol for the patient using the detected neural activity in the patient. Determining the personalized neuromodulation treatment protocol includes determining a neural target in the patient for the personalized neuromodulation treatment protocol. Determining the personalized neuromodulation treatment protocol includes determining optimized neural stimulation parameters for the personalized neuromodulation treatment protocol. The method includes receiving the neural target(s) and the optimized neural stimulation parameters. The neuromodulation treatment protocol is delivered to the patient.

In an aspect of the present disclosure, the optimized neural stimulation parameters are updated iteratively.

### BRIEF DESCRIPTION OF THE DRAWINGS

Various aspects and features of the present disclosure are described hereinbelow with reference to the drawings wherein:

FIG. 1A illustrates a system for personalized neuromodulation according to aspects of the present disclosure;

FIG. 1B illustrates an exemplary personalized neural stimulation device;

FIG. 2 illustrates the neuromodulation target optimization module and the neuromodulation parameters optimization module of the system of FIG. 1;

FIG. 3 is a flow chart of a computer-implemented method of determining an optimal and personalize neuromodulation treatment protocol for a patient employable by the system of FIG. 1;

FIG. 4 is another flow chart of a computer-implemented method of determining an optimal and personalize neuromodulation treatment protocol for a patient employable by the system of FIG. 1;

FIG. 5 is a flow chart of a method of personalized neuromodulation according to aspects of the present disclosure; and

FIG. 6 is a block diagram of an exemplary computer of the system of FIG. 1 according to aspects of the present disclosure.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings).

The devices, systems and methods described herein provide a personalized neuromodulation strategy to offer patient-specific and session-specific neuromodulation treatment for optimized neuromodulation treatment outcome.

A software package or application that may be stored in and executable by a desktop computer, smartphone, tablet computer, laptop computer, or a specialized control module includes a neuromodulation target optimization (NTO) module and a neuromodulation parameter optimization (NPO) module. The terms software package and application may be used interchangeably herein. The software package or application will read brain imaging data including but not limited to datasets observed and/or recorded by an electroencephalogram (EEG) system, a functional magnetic resonance imaging (fMRI) system, or a functional near-infrared spectroscopy (fNIRS) system. The NTO optimizes neuromodulation targets (e.g., specific brain regions, brain structures, nerve clusters or circuits or neural clusters or circuits) and the NPO module optimizes the neuromodulation parameters iteratively to optimize the neuromodulation strategy for optimized neuromodulation treatment outcome in numerous neurological and psychiatric diseases including but not limited to Depression, Parkinson's disease, Epilepsy, Schizophrenia, Bladder control, and Dementia. Iteratively refers to multiple training or treatment sessions in a single day or therapy session or to repeated training and treatment sessions that are conducted over an extended period of time (e.g., months or years) to continually adjust and improve neuromodulation on an individualized basis. Neural structures and circuits change in an individual over time (e.g., as a result of improved neural functioning by treating an underlying disease state), and thus modifying treatment protocols within an individual over time has been found to improve treatment outcomes.

The devices, systems and methods described herein provide a patient-specific and session-specific neuromodulation treatment for optimized neuromodulation treatment outcome. The personalized neuromodulation treatment protocols can include, but are not limited to repetitive transcranial magnetic stimulation (rTMS), transcranial direct current stimulation (tDCS), transcranial electrical stimulation (tES), transcranial focused ultrasound (tFUS), or Deep brain stimulation (DBS), or a combination thereof. These neuromodulation technologies have been found to effectively treat neurological and psychiatric diseases. For example, rTMS is a non-invasive neuromodulation tool approved by the US FDA to treat depression. DBS is also an FDA-approved procedure for treating Parkinson's disease. tDCS is a non-invasive neuromodulation technique that injects weak, direct current via scalp electrodes, and shows effectiveness to treat schizophrenia. To improve the patients' outcome of various neurological and psychiatric diseases, it is important to access the benefits of different stimulation targets, parameters, and devices and understand the patient characteristics that will improve the neuromodulation outcomes.

The devices, systems and methods described herein are employed to identify personalized optimal stimulation targets (e.g., target brain or neural structures) and parameters selection (e.g., stimulation intensity, duration and timing) that can dynamically utilize different neuromodulation technologies, to precisely determine the optimal stimulation targets, amplitudes, and frequencies, and provide precision therapies to treat various neurological and psychiatric diseases. Optimized and personalized target and parameter selection provides a solution for a personalized neuromodulation treatment protocol with minimum dosage for stimulation energy which helps reduce neuromodulation risks to some patients.

The brain controllability analysis (e.g., functional controllability) described in more detail below can determine the optimal stimulation targets for neuromodulation, and can also phenotype various neurological or psychiatric diseases caused by neural control circuit deficits. For example, brain controllability analysis can phenotype and differentiate mild cognitive impairment (MCI) patients who have depressive symptoms from those who do not. The brain controllability analysis can also be employed to phenotype other brain disorders caused by cognitive control or other neural control deficits of the brain, to provide personalized and precise treatments (e.g., neuromodulation or drug treatments) to maximize the treatment efficacy (e.g., by employing personalized neuromodulation or personalized drug treatment protocols). The other brain disorders include, for example, Parkinson's disease, Dystonia, Tremor, Tics associated with Tourette syndrome, Obsessive compulsive disorder, Anxiety, Tinnitus, Gastrointestinal disorder, Hypertension, Angina, Peripheral vascular disorder, Sensory disabilities, Bladder control, Epilepsy, Headache, Chronic pain, Spasticity, Multiple sclerosis, and Stroke. Neuromodulation treatments include, for example, spinal cord stimulation, deep brain stimulation, transcranial magnetic stimulation, transcranial direct current stimulation, a transcranial electrical stimulation, transcranial focused ultrasound stimulation, intracranial cortical stimulation, pressure/mechanical wave (shock wave) stimulation, gastric stimulation, hypoglossal nerve stimulation, peripheral nerve stimulation, retinal stimulation, sacral nerve stimulation, transcranial electrical nerve stimulation, trigeminal nerve stimulation, vagus nerve stimulation, photobiomodulation, or drug treatments such as baclofen infusion, intrathecal drug delivery, or intraventricular drug delivery.

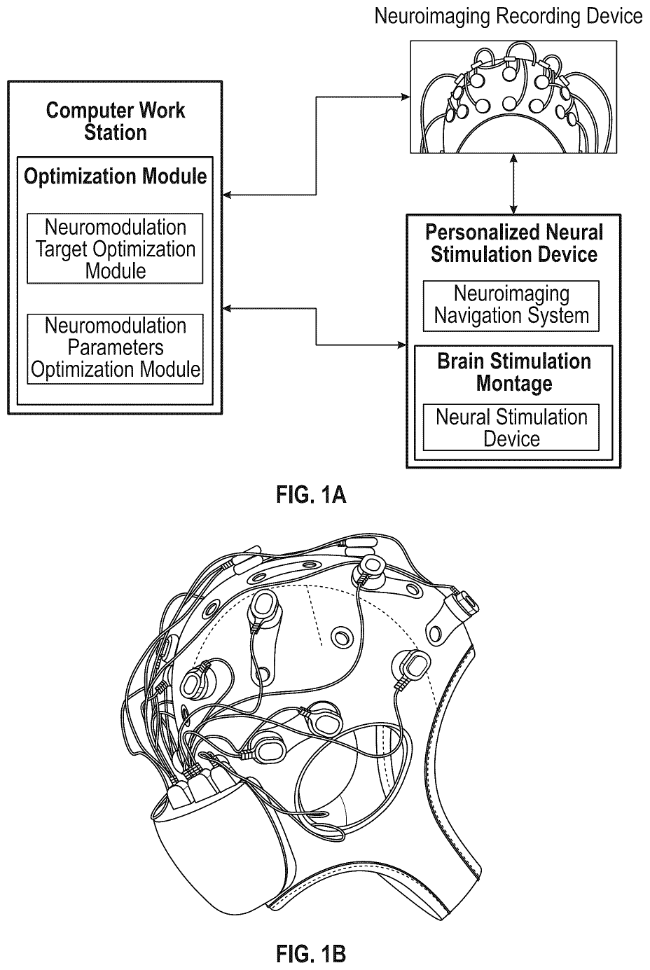

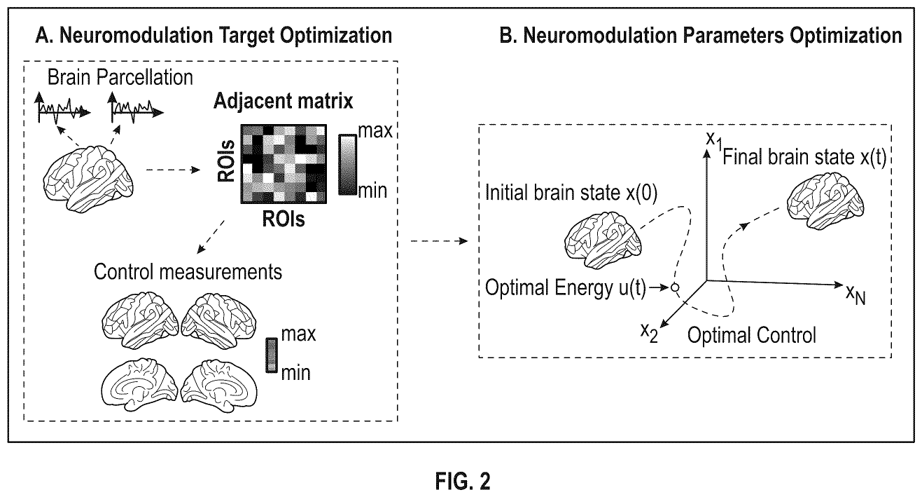

Referring to FIGS. 1A, 1B and 2, a system for personalized neuromodulation including a neuroimaging recording device including sensors for detecting neural activity in a patient. As an example, the neuroimaging recording device may be an electroencephalogram (EEG) system, a functional magnetic resonance imaging (fMRI) system, a diffusion tensor imaging (DTI) system, a positron emission tomography (PET) system, a magnetic resonance spectroscopy (MRS), a Single-photon emission computed tomography (SPECT) system, a functional ultrasound imaging (fUS) system, a computer tomography (CT) system, a functional near-infrared spectroscopy (fNIRS) system, or a magnetoencephalography (MEG) system. The neuroimaging recording device may include wired EEG sensors or wireless EEG sensors.

The neuroimaging recording device communicates (e.g., through a wired or wireless (e.g., WiFi or Bluetooth®) connection) with an optimization module.

The optimization module determines a personalized neuromodulation treatment protocol for the patient based on neural activity data received from the neuroimaging recording device. The optimization module includes a neuromodulation target optimization module (see, e.g., FIG. 2) for determining at least one neural target in the patient for the personalized neuromodulation treatment protocol. The optimization module includes a neuromodulation parameters optimization module (see, e.g., FIG. 2) for determining optimized neural stimulation parameters for the personalized neuromodulation treatment protocol.

A personalized neural stimulation device receives the neural target(s) from the neuromodulation target optimization module and receives the optimized neural stimulation parameters from the neuromodulation parameters optimization module. The personalized neural stimulation device delivers the personalized neuromodulation treatment protocol to the patient. The personalized neural stimulation device includes at least one neural stimulation device for stimulating the target(s) in the patient. A neuroimaging navigation system determines a placement of the neural stimulation device(s) about the patient. The neuroimaging navigation system is employed for guiding ideal positioning of the neural stimulation device on an individual basis (e.g., on a patients head) to account for individual differences in neural anatomy or to account for changes in individual neural structures that occur over time.

As an example, the neural stimulation device includes a transcranial stimulation device. The transcranial stimulation device may be a transcranial magnetic stimulator (TMS), a transcranial direct current stimulator (tDCS), a transcranial electrical stimulator (tES), a transcranial focused ultrasound (tFUS), a deep brain stimulator (DBS), a intracranial cortical stimulation (ICS), a pressure/mechanical wave (shock wave) stimulator, a micro-scale magnetic fields stimulator, an optogenetics stimulator, or a photobiomodulation stimulator.

Referring particularly to the neuromodulation target optimization module of FIG. 2 an exemplary network construction and control set representation is illustrated. The brain was parcellated into N=62 regions of interest (ROIs) using Desikan-Killiany-Tourville atlas. The resting state fMRI signals were extracted from each ROI and utilized to construct the effective brain network and calculate the functional controllability.

Referring particularly to the neuromodulation parameters optimization module of FIG. 2, an exemplary schematic of the optimal control paradigm is illustrated. In the optimal control design, the initial brain state x(0) has some position in space that evolves over time toward a predefined target state x(t). At every time point, the optimal energy (u(t)) required at the stimulating site to propel the system to the target state was calculated.

Referring particularly to FIG. 1B, the personalized neural stimulation device may be a high-density transcranial electrical stimulation (tES) device. The tES device may be capable of running a range of transcranial electrical stimulation paradigms, such as transcranial direct current stimulation (tDCS), transcranial alternating current stimulation (tACS), transcranial random noise stimulation (tRNS), pulsed tDCS, amplitude modulated tACS, or analogue input stimulation modes.

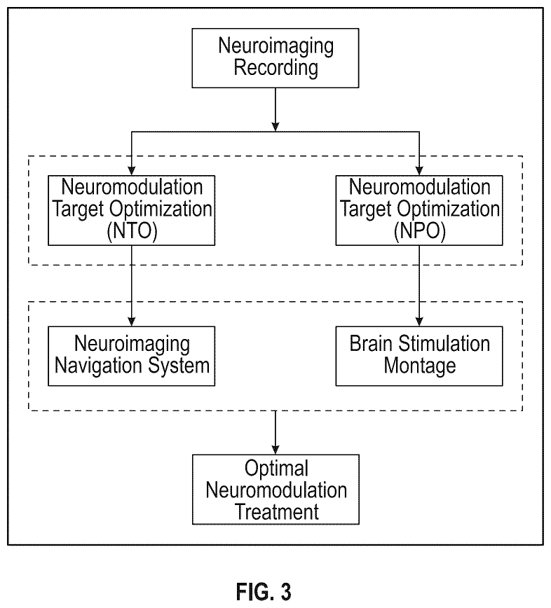

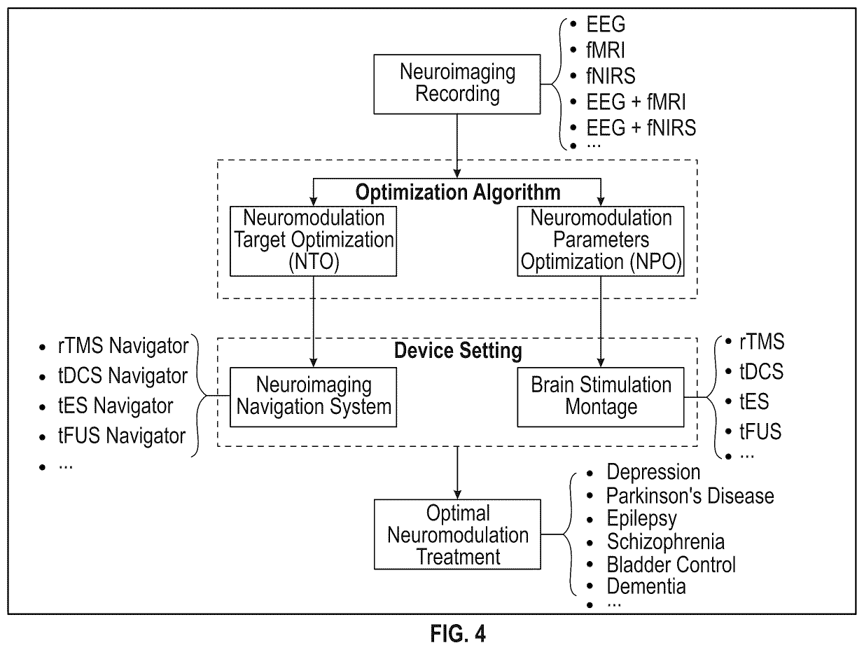

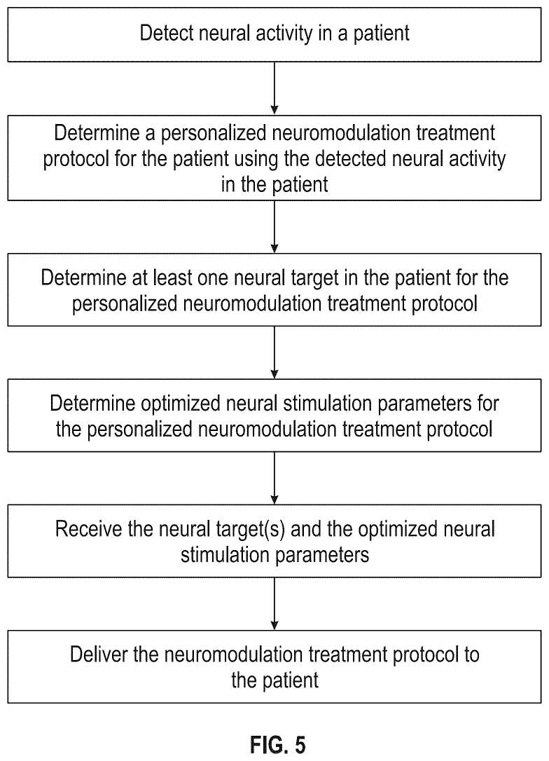

Referring to FIGS. 3 to 5, a method of personalized neuromodulation includes detecting neural activity in a patient (e.g., neuroimaging recording by the neuroimaging recording device). The method includes determining a personalized neuromodulation treatment protocol for the patient (e.g., by the optimization module) using the detected neural activity in the patient. Determining the personalized neuromodulation treatment protocol includes determining a neural target or targets in the patient for the personalized neuromodulation treatment protocol (e.g., by the neuromodulation target optimization module). Determining the personalized neuromodulation treatment protocol includes determining optimized neural stimulation parameters for the personalized neuromodulation treatment protocol (e.g., by the neuromodulation parameters optimization module). The method includes receiving the neural target(s) and the optimized neural stimulation parameters. The neuromodulation treatment protocol is delivered to the patient.

In an aspect of the present disclosure, the optimized neural stimulation parameters are updated iteratively. Iteratively may refer to multiple updates during a single treatment section, or changes to the personalized neuromodulation treatment protocol over different treatment sessions.

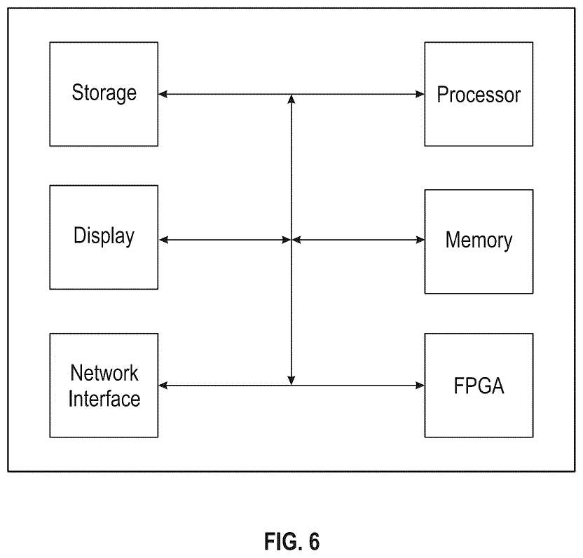

The method described above with reference to FIGS. 3 to 5, may be a computer-implemented method (e.g., a method performed by the computer described in more detail below with reference to FIG. 6).

An exemplary algorithm employable, for example, by the system described above with reference to FIG. 1 includes reading brain imaging data including but not limited to f/MRI, EEG, and f/NIRS datasets. Once the neuroimaging datasets are obtained, the data is read into the NTO module of the software package or application for preprocessing, region of interest (ROI) analysis, and brain network construction. Based on the brain network, brain controllability analysis is performed to calculate the controllability of each single brain region, and then the stimulation targets are determined from the brain regions with the highest controllability values. A simulation process is then conducted in the NPO module of the software package or application, to determine the optimal stimulation parameters including stimulation amplitudes and frequencies, to steer the diseased brain states of patients to the healthy level. The optimal stimulation targets and parameters are then sent to the neuroimaging navigation system and the brain stimulator, respectively, to modulate the brain states of patients.

The exemplary algorithm can employ the following steps:

Step 1: record the neuroimaging signals from the patients. EEG, f/MRI, and f/NIRS are the main neuroimaging modalities that are employed to collect the patients' neurophysiological signals on the brain. The neuroimaging data will is as a Brain Imaging Data Structure (BIDS), a standard for organizing and describing the neuroimaging datasets, before exporting to the software package or application (e.g., in a same computer or control module workstation). The BIDS standard uses file formats compatible with existing software, unifies the majority of practices already common in the field, and captures the metadata necessary for most common data processing operations.

Step 2: Once the neuroimaging signals are recorded from patients, the BIDS datasets are exported to the NTO module of the software package or application (e.g., in the same workstation), to identify the optimal stimulation targets. In the NTO module, several sub-modules re built including a preprocessing module to remove the physiological signal artifacts, region of interest (ROI) analysis module to perform the brain segmentation and calculate the activities of each brain region, and brain network construction module to build the brain networks that reflect the connectivity between different brain areas. Based on the brain networks constructed by the above steps, the main sub-module, brain network controllability (BNC) calculation module is run to compute the controllability of each single brain region. The network controllability analysis has recently been developed to describe the capability of different brain areas in controlling the functional brain network states based on the control theory. The BNC calculation module employs an advanced network dynamic model, where the rate of state changes are determined by the current state and a random diffusion process. Mathematically, the network dynamic model can be described as:

$$d {x}_{i} = ( - \frac{1}{{τ}_{i}} + {\sum }_{j \neq i} {C}_{ij} ) * {x}_{i} dt + σ * {dB}_{i}$$

where xᵢ is the brain state of region i, τᵢ is the time constant indicating state decay over time, Cᵢⱼ represents the brain connectivity matrix between region i and j constructed from the functional brain networks, and (σ*dBᵢ) is formally a Wiener process with covariance σ. To fit the dynamics, the unknown parameters τᵢ, Cᵢⱼ, and σ are estimated by minimizing the loss between the model-derived and empirical covariance matrices using the Lyapunov optimization method.

$$Let A = ( - \frac{1}{{τ}_{i}} + {\sum }_{j \neq i} {C}_{ij} ) and B = σ * {dB}_{i} ,$$

the controllability c is then calculated as:

$$c = \sqrt{trace [ {B}^{T} ( {\int }_{0}^{+ ∞} exp ( At + {A}^{T} t ) dt ) B ]}$$

The brain regions with the maximum controllability values are selected as reference to determine the optimal stimulation targets.

Step 3: after the optimal stimulation targets are located in the NTO module, the NPO module can be run immediately, to calculate the optimal stimulation parameters, including the stimulation amplitudes and frequencies, to steer the diseased brain state of patients to the healthy level. The parameters optimization problem will be defined as:

$${min}_{u} {\int }_{0}^{T} {( {x}_{T} - x ( t ) )}^{′} S ( {x}_{T} - x ( t ) ) + ρ {{u}_{K} ( t )}^{′} {u}_{K} dt$$

$$s . t . \dot{x} = A x ( t ) + Bu ( t ) , x ( 0 ) = {x}_{0} , and x ( T ) = {x}_{T}$$

where x is the current state, x_{T} is the target state, T is the control horizon, a free parameter that defines the finite amount of time to reach the target state, and p is a free parameter that weights the input constraint. S is the set of nodes to constrain. With these definitions, two constraints emerge from the optimization problem. First, (x_{T}−x(t))′S(x_{T}−x(t)) constrains the trajectories of a subset of nodes by preventing the system from traveling too far from the target state. Second, ρu_{K}(t)′u_{K} constrains the amount of input utilized to reach the target state, a requirement for biological systems, which are limited by metabolic demands and tissue sensitivities.

Step 4: after the optimal stimulation targets and parameters are determined by our software package or application, the targets for stimulation are identified by combining MRI images of patients with areas identified as optimal stimulation targets by the neuronavigation system (e.g., in the same workstation). The neuronavigation system can provide feedback on coil position and coil orientation (trajectory: pitch, tilt, and yaw) with respect to these predefined targets, critical in accurately repositioning the coil across multiple sessions. By employing neuronavigation technologies, the accuracy, reliability and repeatability of positioning the neural stimulation device is maximized, thus resulting in a personalized neural stimulation device configured to apply the personalized neuromodulation treatment protocol for the patient. After the stimulation targets are optimally located by the neuronavigation system, the stimulation amplitudes and frequencies will be set to the neural stimulation devices. The optimal stimulation parameters inferred from the optimization algorithm are the time-course of the stimulation amplitudes or frequencies. Thus, the time-course stimulation parameters are set as a customized stimulation montage to perform optimal neuromodulation on a personalized basis.

All modules described herein may be integrated into one workstation, including neuroimaging recording module, the software package or application (including NTO and NPO modules), neuroimaging navigation system, and brain stimulation module. The integrated system can employ a quantum computer to share determined protocols via a server to other parties, such as researchers or clinicians.

FIG. 6 is a block diagram of an exemplary computer of the system of FIG. 1 according to aspects of the present disclosure.

Referring to FIG. 6, the computer may include a processor connected to a computer-readable storage medium or a memory which may be a volatile type memory, e.g., RAM, or a non-volatile type memory, e.g., flash media, disk media, etc. The processor may be another type of processor such as, without limitation, a digital signal processor, a microprocessor, an ASIC, a graphics processing unit (GPU), field-programmable gate array (FPGA), or a central processing unit (CPU).

In some aspects of the disclosure, the memory can be random access memory, read-only memory, magnetic disk memory, solid state memory, optical disc memory, and/or another type of memory. The memory can communicate with the processor through communication buses of a circuit board and/or through communication cables such as serial ATA cables or other types of cables. The memory includes computer-readable instructions that are executable by the processor to operate the control module/control unit, computer or workstation. The computer may include a network interface (e.g., a wireless network interface) to communicate with other computers or a server. A storage device may be used for storing data. The computer may include one or more FPGAs. The FPGA may be used for executing various machine learning algorithms.

In an aspect of the present disclosure, the computer is wirelessly connected with the neuroimaging recording device and/or the personalized neural stimulation device. Alternatively, the computer may be connected with the neuroimaging recording device and/or the personalized neural stimulation device via a wired connection (e.g., a USB connection). As an example, the computer may be included in a smartphone or tablet computer. The computer may also be a laptop or desktop computer in communication with the neuroimaging recording device and/or the personalized neural stimulation device. The computer may also be housed in a special purpose control device or workstation (see, e.g., FIG. 1).

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A system for personalized neuromodulation, comprising:
a neuroimaging recording device including a plurality of sensors configured to detect neural activity in a patient;
an optimization module configured to receive neural activity data from the neuroimaging recording device and determine a personalized neuromodulation treatment protocol for the patient, the optimization module including:
a neuromodulation target optimization module configured to determine at least one neural target in the patient for the personalized neuromodulation treatment protocol; and
a neuromodulation parameters optimization module configured to determine optimized neural stimulation parameters for the personalized neuromodulation treatment protocol; and
a personalized neural stimulation device configured to receive the at least one neural target from the neuromodulation target optimization module and to receive the optimized neural stimulation parameters from the neuromodulation parameters optimization module,
the personalized neural stimulation device configured to deliver the personalized neuromodulation treatment protocol to the patient, the personalized neural stimulation device including:
a neural stimulation device configured to stimulate the at least one target in the patient; and
a neuroimaging navigation system configured to determine a placement of the at least one neural stimulation device about the patient.

2. The system for personalized neuromodulation of claim 1, wherein the neural stimulation device includes a transcranial stimulation device.

3. The system for personalized neuromodulation of claim 1, wherein the neural stimulation device is a transcranial magnetic stimulator (TMS), a transcranial direct current stimulator (tDCS), a transcranial electrical stimulator (tES), a transcranial focused ultrasound (tFUS), a deep brain stimulator (DBS), a intracranial cortical stimulation (ICS), a pressure/mechanical wave (shock wave) stimulator, a micro-scale magnetic fields stimulator, an optogenetics stimulator, or a photobiomodulation stimulator.

4. The system for personalized neuromodulation of claim 1, wherein the neuroimaging recording device is an electroencephalogram (EEG) system, a functional magnetic resonance imaging (fMRI) system, a diffusion tensor imaging (DTI) system, a positron emission tomography (PET) system, a magnetic resonance spectroscopy (MRS), a Single-photon emission computed tomography (SPECT) system, a functional ultrasound imaging (fUS) system, a computer tomography (CT) system, a functional near-infrared spectroscopy (fNIRS) system, or a magnetoencephalography (MEG) system.

5. The system for personalized neuromodulation of claim 1, wherein the neuroimaging recording device includes wired EEG sensors.

6. The system for personalized neuromodulation of claim 1, wherein the neuroimaging recording device includes wireless EEG sensors

7. A method of personalized neuromodulation, comprising:
detecting, by a neuroimaging recording device including a plurality of sensors, neural activity in a patient;
determining, by an optimization module, a personalized neuromodulation treatment protocol for the patient using the detected neural activity in the patient, wherein determining the personalized neuromodulation treatment protocol includes:
determining, by a neuromodulation target optimization module, at least one neural target in the patient for the personalized neuromodulation treatment protocol; and
determining, by a neuromodulation parameters optimization module, optimized neural stimulation parameters for the personalized neuromodulation treatment protocol;
receiving, at a personalized neural stimulation device, the at least one neural target from the neuromodulation target optimization module and the optimized neural stimulation parameters from the neuromodulation parameters optimization module of the neuromodulation treatment protocol; and
delivering, by the personalized neural stimulation device, the neuromodulation treatment protocol to the patient.

8. The method of personalized neuromodulation of claim 7, wherein the personalized neural stimulation device includes a neural stimulation device configured to stimulate the at least one target in the patient; and wherein a placement of the neural stimulation device is determined by a neuroimaging navigation system configured to determine a placement of the at least one neural stimulation device about the patient.

9. The method of personalized neuromodulation of claim 8, wherein the neural stimulation device includes a transcranial stimulation device positioned about the patient.

10. The method of personalized neuromodulation of claim 8, wherein the neural stimulation device includes a transcranial magnetic stimulator (TMS), a transcranial direct current stimulator (tDCS), a transcranial electrical stimulator (tES), a transcranial focused ultrasound (tFUS), a deep brain stimulator (DBS), a intracranial cortical stimulation (ICS), a pressure/mechanical wave (shock wave) stimulator, a micro-scale magnetic fields stimulator, an optogenetics stimulator, or a photobiomodulation stimulator positioned about the patient.

11. The method of personalized neuromodulation of claim 7, wherein the neuroimaging recording device includes an electroencephalogram (EEG) system, a functional magnetic resonance imaging (fMRI) system, a diffusion tensor imaging (DTI) system, a positron emission tomography (PET) system, a magnetic resonance spectroscopy (MRS), a Single-photon emission computed tomography (SPECT) system, a functional ultrasound imaging (fUS) system, a computer tomography (CT) system, a functional near-infrared spectroscopy (fNIRS) system, or a magnetoencephalography (MEG) system.

12. The method of personalized neuromodulation of claim 7, wherein the neuroimaging recording device includes wired EEG sensors positioned about the patient.

13. The method of personalized neuromodulation of claim 7, wherein the neuroimaging recording device includes wireless EEG sensors positioned about the patient.

14. The method of personalized neuromodulation of claim 7, wherein the optimized neural stimulation parameters are updated iteratively.

15. A computer program product comprising a non-transitory computer readable storage medium having program instructions embodied therewith, the program instructions executable by a processor to cause the processor to:
detect, by a neuroimaging recording device including a plurality of sensors, neural activity in a patient;
determine, by an optimization module, a personalized neuromodulation treatment protocol for the patient using the detected neural activity in the patient, wherein determining the personalized neuromodulation treatment protocol includes:
determining, by a neuromodulation target optimization module, at least one neural target in the patient for the personalized neuromodulation treatment protocol; and
determining, by a neuromodulation parameters optimization module, optimized neural stimulation parameters for the personalized neuromodulation treatment protocol;
receive, at a personalized neural stimulation device, the at least one neural target from the neuromodulation target optimization module and the optimized neural stimulation parameters from the neuromodulation parameters optimization module of the neuromodulation treatment protocol; and
deliver, by the personalized neural stimulation device, the neuromodulation treatment protocol to the patient.
